(19) United States
(12) Patent Application Publication
(10) Pub. No.: US 2020/0229481 A1
(43) Pub. Date: Jul. 23, 2020

(54) Methods of Providing FODMAP Friendly Food Items

(71) Applicant: Ketan Vakil, New York, NY (US)

(72) Inventor: Ketan Vakil, New York, NY (US)

(21) Appl. No.: 16/250,881

(22) Filed: Jan. 17, 2019

(51) Int. Cl. A23L 33/00; A23L 33/21; G16H 20/60

(52) U.S. Cl. CPC A23L 33/40; A23V 2002/00; G16H 20/60; A23L 33/21

(57) ABSTRACT

A method of creating a FODMAP diet friendly food is described. The method includes identifying a recipe for a food item containing at least one FODMAP diet unfriendly food ingredient. The method also includes determining substitutes and a quantity of the substitutes for the FODMAP diet unfriendly food ingredient to substantially maintain the taste profile of the food item created by the recipe. The substitutes include a FODMAP diet friendly garlic substitute and a FODMAP diet friendly onion substitute. The method further includes providing and preparing the substitutes for inclusion in the recipe.

### BACKGROUND

Digestive-related conditions affect more than 70 million people in the United States and account for 48.3 million ambulatory care visits each year—identifying food that they can safely eat without triggering a flare up is a source of deep frustration.

Restricting certain foods can dramatically improve these symptoms in sensitive people. A diet low in fermentable carbs known as FODMAPS is clinically recommended for the management of irritable bowel syndrome (IBS). For those that are intolerant, the benefits of a low-FODMAP or FODMAP friendly diet may include less gas, less bloating, less diarrhea, less constipation and less stomach pain.

FODMAP stands for fermentable oligo-, di-, mono-saccharides and polyols. FODMAPs are found in a wide range of foods in varying amounts. Some foods contain just one type, while others contain several. These are short-chain carbs that are resistant to digestion. Instead of being absorbed into your bloodstream, they reach the far end of your intestine where most of your gut bacteria reside. Your gut bacteria then use these carbs for fuel, producing hydrogen gas and causing digestive symptoms in sensitive individuals. FODMAPs also draw liquid into your intestine, which may cause diarrhea. Although not everyone is sensitive to FODMAPs, this is very common among people with irritable bowel syndrome (IBS)

The oligo category combines two common oligosaccharides that are poorly absorbed and can trigger IBS symptoms, the fructans and galacto-oligosaccharides. In order to absorb fructans, we need to break the sugars down into single sugars (monosaccharides). But, we can't. Humans do not produce any enzymes that can break down the bonds between these sugars, so the fructans move through the gut completely unabsorbed. When the fructans meet bacteria in the large intestine, they are fermented which produces gases.

In healthy people this causes a bit of wind and is part of normal, healthy digestion. In IBS, where the gut is hypersensitive and motility disturbances are common, this results in bloating, abdominal discomfort and altered motility. These oligosaccharides (oligos) always pass through the gut and escape digestion. They have beneficial effects whereby they encourage the growth of good bacteria and act as prebiotics, but for those that have IBS they may notice symptoms after ingesting foods rich in the oligos.

Accordingly, there is a need for FODMAP friendly foods which are low in the oligos to help those with IBS symptoms. There is further a need for FODMAP friendly foods which do not sacrifice flavor in order to be low in FODMAP ingredients.

### SUMMARY

An exemplary embodiment relates to a method of creating a FODMAP diet friendly food. The method includes providing a recipe for a food item containing at least one of onion and garlic. The method also includes determining substitutes and a quantity of the substitutes for the onion and garlic to substantially maintain the taste profile of the food item created by the recipe. The substitutes include garlic scapes, spring garlic tops and a FODMAP diet friendly onion substitute. Further, the method includes providing and preparing the substitutes for inclusion in the recipe.

Another exemplary embodiment relates to a method of creating a FODMAP diet friendly food. The method includes providing a recipe for a food item containing at least one of onion and garlic. The method also includes determining substitutes and a quantity of the substitutes for the onion and garlic to substantially maintain the taste profile of the food item created by the recipe. The substitutes include a FODMAP diet friendly garlic substitute and at least one of green tops of leeks, green spring onion tops, chives, and the green tops of scallions. Further, the method includes providing and preparing the substitutes for inclusion in the recipe.

Yet another exemplary embodiment relates to a method of creating a FODMAP diet friendly food. The method includes identifying a recipe for a food item containing at least one FODMAP diet unfriendly food ingredient. The method also includes determining substitutes and a quantity of the substitutes for the FODMAP diet unfriendly food ingredient to substantially maintain the taste profile of the food item created by the recipe. The substitutes include a FODMAP diet friendly garlic substitute and a FODMAP diet friendly onion substitute. The method further includes providing and preparing the substitutes for inclusion in the recipe.

In addition to the foregoing, other system aspects are described in the claims, drawings, and text forming a part of the disclosure set forth herein. The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the disclosures set forth herein.

### BRIEF DESCRIPTION OF THE DRAWINGS

FIG. 1 is an exemplary embodiment process FODMAP friendly food items.

The drawings are merely exemplary of one of many possible processes falling under the scope of the invention and should not be viewed as limiting.

### DETAILED DESCRIPTION

Most packaged foods (sauces, soups, spice mixes, salsas, dressings, condiments, etc) contain high levels of fructans in the form of onion and garlic. These are very common flavor enhancers.

Conventionally, there are products on the market which substantially remove these and other high-fodmap ingredients however, the flavors that those ingredients impart are not readily replaced.

In accordance with an exemplary embodiment a solution is to replace onion and garlic with different parts of plants that are often little known and/or often thrown away to retain true garlic and onion flavor while ensuring the packaged foods remain low FODMAP or fodmap diet friendly. For example, the green shoots that grow out of onions (green spring onions, ramp leaves) and the flower buds of garlic (garlic scapes) are low in fructans, as are all manner of chives, the green tops of leeks, the green tops of young “spring” garlic and the green tops of scallions. In accordance with the exemplary method, the method replaces the garlic and onion bulbs with these items, crafting soups, spices and sauces with similar flavor that are gut-friendly for those following the low FODMAP diet.

In accordance with an exemplary embodiment, a food product designer may take a recipe having FODMAP unfriendly foods mixed in, and figure out substitutes for those FODMAP unfriendly foods without making great sacrifice as to taste. The substitutes may come from the groups of foods which are FODMAP friendly but retain some of the removed flavors.

For example, a Taco spice blend conventionally includes powdered or dehydrated onion and powdered or dehydrated garlic. In accordance with an exemplary embodiment, a substitution of Freeze Dried Green Onion and Freeze Dried Ground Garlic Scape is substituted therefor. An ingredient list of such an exemplary FODMAP friendly taco spice blend is provided below.

| Ingredient | Notes |
|---|---|
| Pure New Mexican Chile Powder | |
| Cumin seed | Toasted and ground |
| Salt | |
| Coriander seed | Toasted and ground |
| Freeze Dried Green Onion | Flakes toasted and ground |
| Freeze Dried Garlic Scape | Flakes toasted and ground |
| Hungarian Paprika | |
| Black Pepper | |
| Indio Oregano | Flakes toasted and ground |
| Red Pepper Flake | Flakes toasted and ground |

As another example, a chicken stock conventionally includes fresh onion and garlic. In accordance with an exemplary embodiment, a substitution of fresh green onion tops, garlic scapes, green leek tops and chives are substituted therefor. An ingredient list of such an exemplary FODMAP friendly chicken broth is provided below.

| Ingredients | Notes |
|---|---|
| Chicken bones | |
| Spring onions | Only dark green areas |
| Garlic scapes | Green areas |
| Bay leaves | |
| Carrots | |
| Leeks | Dark green tops only |
| Celery | |
| Celery leaves | |
| Chives | |
| Parsley leaves | |
| Water | |

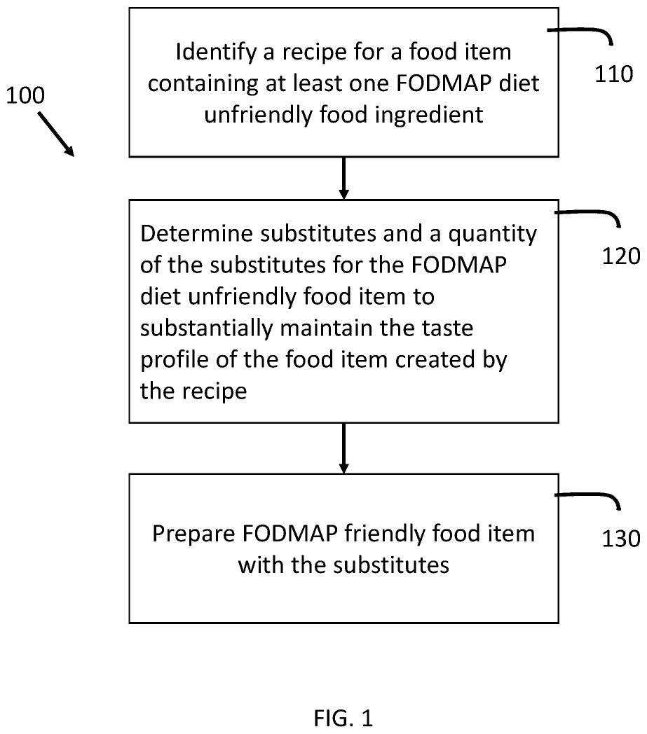

Referring now to FIG. 1, a process of for creating a FODMAP friendly food 100 is depicted. The process includes identifying a recipe for a food item containing at least one FODMAP diet unfriendly food ingredient (Process Step 110). Once the FODMAP diet unfriendly food ingredients are identified, it is beneficial to determine substitutes for those FODMAP diet unfriendly food ingredients which also tend to substitute the flavors being lost by removing FODMAP diet unfriendly food ingredients (Process Step 120). This may require a great deal of trial and error and taste testing in order to accomplish similar flavors. Once the substitutes have been determined, the FODMAP diet friendly food items may be prepared in accordance with the adapted recipe containing the substitutes (Process Step 130).

There are many ways in which the FODMAP friendly ingredients may be incorporated into the recipe. These ways include but are not limited to chopping the substitutes, pulverizing the substitutes such that the substitutes produce a slurry or paste, deriving essential oils from the substitutes, cooking the substitutes, dehydrating or freeze drying the substitutes, boiling or simmering the substitutes, etc. Providing the correct manner in which to incorporate the substitutes will go to producing the desired taste profile.

In accordance with an exemplary embodiment of the invention, it is desirable to provide substitutes for both the onion and the garlic in the FODMAP diet unfriendly recipes. The garlic and onions are often some of the most FODMAP unfriendly foods. The garlic and onion may be substituted by one or a combination of the following food ingredients: green spring onion tops, chives, the green tops of leeks, the green tops of scallions, the green tops of young garlic, ramp leaves, and garlic scapes, among others.

In an exemplary embodiment it may be that only the onion is getting removed and replaced from a FODMAP diet unfriendly recipe. In this case a food designer may choose especially from green spring onion tops, chives, the green tops of leeks, and the green tops of scallions, to maintain a substantially similar flavor profile.

Likewise, in an exemplary embodiment it may be that only the garlic is getting removed and replaced from a FODMAP diet unfriendly recipe. In this case a food designer may choose especially from ramp leaves, leek tops, garlic chives, green/spring garlic tops and garlic scapes.

In some exemplary embodiments it may be chosen that a food designer may generate a recipe from scratch having the chosen onion and garlic substitutes that are FODMAP diet friendly instead of ever using the traditional parts of onions and/or garlic while at the same time attempting to achieve a flavor profile which reads as containing onions and/or garlic.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

What is claimed is:

1. A method of creating a FODMAP diet friendly food, comprising:
providing a recipe for a food item containing at least one of onion and garlic;
determining substitutes and a quantity of the substitutes for the onion and garlic to substantially maintain the taste profile of the food item created by the recipe, the substitutes including at least one of ramp leaves, leek tops, garlic chives, green young garlic tops and garlic scapes and a FODMAP diet friendly onion substitute; and
providing and preparing the substitutes for inclusion in the recipe.

2. The method of claim 1, wherein preparing the substitutes includes chopping the substitutes.

3. The method of claim 1, wherein preparing the substitutes includes pulverizing the substitutes into a slurry or paste.

4. The method of claim 1, wherein preparing the substitutes includes removing the essential oils from the FODMAP diet friendly onion substitute.

5. The method of claim 1, wherein the FODMAP diet friendly onion substitute includes green spring onion tops.

6. The method of claim 1, wherein the FODMAP diet friendly onion substitute includes chives.

7. The method of claim 1, wherein the FODMAP diet friendly onion substitute includes the green tops of leeks.

8. The method of claim 1, wherein the FODMAP diet friendly onion substitute includes the green tops of scallions.

9. A method of creating a FODMAP diet friendly food, comprising:
providing a recipe for a food item containing at least one of onion and garlic;
determining substitutes and a quantity of the substitutes for the onion and garlic to substantially maintain the taste profile of the food item created by the recipe, the substitutes including a FODMAP diet friendly garlic substitute and at least one of green tops of leeks, green spring onion tops, chives, and the green tops of scallions; and
providing and preparing the substitutes for inclusion in the recipe.

10. The method of claim 9, wherein preparing the substitutes includes chopping the substitutes.

11. The method of claim 9, wherein preparing the substitutes includes pulverizing the substitutes into a slurry or paste.

12. The method of claim 9, wherein preparing the substitutes includes removing the essential oils from the FODMAP diet friendly garlic substitute.

13. The method of claim 1, wherein the FODMAP diet friendly garlic substitute includes ramp leaves.

14. The method of claim 1, wherein the FODMAP diet friendly substitute includes garlic scapes.

15. A method of creating a FODMAP diet friendly food, comprising:
identifying a recipe for a food item containing at least one FODMAP diet unfriendly food ingredient;
determining substitutes and a quantity of the substitutes for the FODMAP diet unfriendly food ingredient to substantially maintain the taste profile of the food item created by the recipe,
the substitutes including a FODMAP diet friendly garlic substitute and a FODMAP diet friendly onion substitute; and
providing and preparing the substitutes for inclusion in the recipe.

16. The method of claim 15, wherein preparing the substitutes includes chopping the substitutes.

17. The method of claim 15, wherein preparing the substitutes includes pulverizing the substitutes into a slurry or paste.

18. The method of claim 15, wherein preparing the substitutes includes removing the essential oils from the FODMAP diet friendly substitutes.

19. The method of claim 15, wherein the FODMAP diet friendly garlic substitute includes at least one of ramp leaves, leek tops, garlic chives, green garlic tops, and garlic scapes.

20. The method of claim 15, wherein the FODMAP diet friendly onion substitute includes at least one of green spring onion tops, chives, green tops of leeks, and green tops of scallions.
